(19) United States
(12) Patent Application Publication
(10) Pub. No.: US 2005/0202043 A1
(43) Pub. Date: Sep. 15, 2005

(54) Multimerised HIV fusion inhibitors

(75) Inventor: Michael Etzerodt, Hinnerup (DK)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Borean Pharma A/S, Aarhus C (DK)

(21) Appl. No.: 11/064,115

(22) Filed: Feb. 23, 2005

Related U.S. Application Data
(60) Provisional application No. 60/546,200, filed on Feb. 23, 2004.

(30) Foreign Application Priority Data
Feb. 23, 2004 (DK) PA 2004 00283
Feb. 23, 2005 (WO) PCT/DK05/00121

(51) Int. Cl. C12Q 1/70; A61K 39/21; C07K 14/16

(52) U.S. Cl. 424/208.1; 435/5; 530/350

(57) ABSTRACT

There are provided multimeric fusion proteins exhibiting anti-viral activity. The fusion proteins comprise the HR2 region of the ectodomain of the human immunodeficiency virus gp41 protein which is fused to a multimerisation domain peptide such as a trimerisation domain derived from tetranectin. The multimerised fusion proteins may be used as HIV fusion inhibitors in the treatment of AIDS.

### CROSS-REFERENCE TO RELATED APPLICATIONS

This application claims the benefit of PCT Application No.______, filed Feb. 23, 2005, which claims the benefit of U.S. Provisional Application No. 60/546,200, filed Feb. 23, 2004, and Denmark Patent Application No. PA 2004 00283, filed Feb. 23, 2004. This application also claims the benefit of U.S. Provisional Application No. 60/546,200, filed Feb. 23, 2004 and Denmark Patent Application No. PA 2004 00283, filed Feb. 23, 2004. The disclosures of the aforementioned applications are herein incorporated by reference in their entireties.

### TECHNICAL FIELD

The present invention relates to multimeric fusion proteins exhibiting anti-viral activity which comprises the HR2 region of the ectodomain of the human immunodeficiency virus gp41 protein fused to a multimerisation domain peptide. The multimerised fusion proteins may be used as HIV fusion inhibitors in the treatment of AIDS.

### BACKGROUND ART

In 2003 in the US and Western Europe approximately 1.2 million people were infected with Human Immunodeficiency Virus, HIV. About 50% of this infected population has already been diagnosed with Acquired Immune Deficiency Syndrome, AIDS. This number has risen linearly from around 200,000 with AIDS in 1993 and with a doubling time of approximately 8 years.

No cure to HIV infection has been established yet—primarily because the virus (as a population), in effect, is capable of escaping the immune system and eventually ends up destroying it. However, a number of drugs, which slow down disease progression, have been developed resulting in significant increases in expected life spans of people infected. This and the fact that HIV infection is chronic, together with a high frequency of mutation and genetic recombination in the process of reverse transcription of the viral genome, result in a steady demand for development of new drugs fighting virus replication, integration, production and infection at more stages in the viral life cycle and/or using new strategies.

HIV is a retrovirus. The viral genome is a single stranded RNA molecule with a genome of around 9 kb. Two RNA copies are present in one viral particle. Upon release into the host cell the viral genome is reverse transcribed, by the virally encoded reverse transcriptase, into a double stranded circular DNA form, which integrates into the cell chromosome as a so-called provirus.

A number of RNAs are transcribed from the provirus. Among them are: a full length copy which encodes the gag and gag-pol poly-proteins and a spliced version which encodes the env poly-protein. Both these polyproteins are processed by proteases during particle maturation. The lentiviruses, the subgroup of retroviruses HIV belongs to, further synthesize a number of other splice variants of mRNAs which encode proteins involved in expression regulation of both the virus and the host cell (tat, nef, rev etc.).

The drugs typically used in the clinic include reverse transcriptase small molecule inhibitors and inhibitors of the viral poly-protein processing protease. Recently a so-called fusion inhibitor, the polypeptide T-20 (also known as Enfuvirtide, Fuzeon and DP-178) has been approved for clinical use. For more detailed information on T-20 please cf. U.S. Pat. No. 5,464,933, U.S. Pat. No. 6,133,418 and U.S. Pat. No. 6,475,491.

This drug belongs to a class of experimental drugs focusing on preventing the virus from infecting the target cell. The T-20 peptide, which is a 36 amino acid residue synthetic polypeptide, and related derivatives (e.g. T1249, C-34 and others), which binds to the viral env-protein gp41 after attachment of the virus to the target cell membrane and prevent infection by inhibition of fusion of the viral envelope membrane and the target cell membrane, has demonstrated a potential in entry inhibition, and pointed to gp41 as a target for development of new anti-HIV drugs. Briefly, infection of a target cell by HIV typically requires interaction of the viral env protein gp120 with two cellular receptors (recently reviewed in Moore et al., 2003). The viral spike-protein complex which represents the major component of the surface of the viral particle, is initially synthesized by the host cell as a transmembrane polyprotein, gp160 of approximately 850 amino acid residues. Gp160 is cleaved into the two env proteins gp120 and gp41 during virus budding and maturation. The spike is organized as a gp41 dependent trimeric complex comprised by three 1:1 non-covalently linked gp120 and gp41 protomers. Initially the HIV particle attach to the target T-cell via binding to a primary receptor, typically the CD4 receptor. Binding to CD4 triggers a conformational change which exposes a previously hidden binding site for either of the chemokine co-receptors CCR5 or CXCR4. Gp102 binding to the co-receptor result in additional conformational changes in the gp120-gp41 complex and the N-terminal part of gp41, the so-called fusion peptide (FP), is displaced towards the target cell membrane and inserts into it. These conformational changes and rearrangements of protein complexes is considered at least partially driven by formation of a triple helical coil-coil organization of the gp41 ectodomain, mediated by the gp41 HR1 region. The trimeric gp41 subunit then folds back on itself, allowing the HR2 region, a second more C-terminal helix forming region in gp41, to pack into groves on the outside of the HR1 tripple-stranded coiled coil. Eventually, a six-helix bundle structure is formed comprised by three central HR1 regions and with three HR2 regions packing on the outside in an anti-parallel orientation. As a result of this structural transition the viral and target cell membranes are brought into close proximity, and the associated change in free energy is considered sufficient to cause the membrane fusion and liberation of the viral core into the target cell cytoplasm.

The mechanism of action of the T-20 peptide and related polypeptide fusion inhibitors is to bind the gp41 HR1 region upon its exposure after the initial viral receptor binding and the associated rearrangements of the env protein complex thereby inhibiting formation of the six-helix bundle structure and eventually preventing membrane fusion and infection of the target cell. The T-20 peptide and the related polypeptide fusion inhibitors are derived from the amino acid sequence of the HR2 region of the ectodomain of gp41. The HR2 region is shown in SEQ ID NO 1. Basically, the ectodomain of gp41 (FIG. 1) (amino acid residues 1 to 174 of the gp41 sequence) comprises the amino acid residues 511 to 685 of the env polyprotein of the HIV LAI clade followed by the amino acid sequence specifying the envelope transmembrane region of gp41. The fusion peptide (FP) which is inserted into the membrane of the target cell is located in the amino terminal part of the gp41 ectodomain followed by the first and second helix regions, HR1 (around amino acid residue 28 to 82) and HR2 (around residue 100 to 170), respectively. The helix forming regions are interspaced by an amino acid sequence characterized by the presence of a disulfide bridge (the so-called disulfide bridge containing region, amino acid residues 83 to 99). The envelope transmembrane region is located immediately C-terminal to the HR2 region.

T-20 and the other known polypeptide HIV target cell membrane fusion inhibitors are all manufactured by standard solid phase peptide synthesis technology. One inhibitor, the five helix bundle protein, is manufactured as a 28 kDa fusion protein produced in E. coli comprising a (HR1)-linker-(HR2)-linker-(HR1)-linker-(HR2)-linker-(HR1) architecture, where (HR1) and (HR2) denote amino acid residue sequences representing partial HR1 and HR2 regions (Root et al., 2001).

Although the T-20 peptide and other HR2 derived synthetic polypeptides have demonstrated a potential in fusion inhibition and T-20 has been approved in several countries for the treatment of HIV infected individuals, it is well known that a number of problems and shortcomings have been identified, both related to the synthetic peptide itself and to the way it is manufactured.

One of these technical problems is the relatively short plasma half-life of T-20. The observed plasma half-life in humans of the compound is approximately 4 hours (3.8±0.6 h; Fuzeon label, Roche Pharmaceuticals, 2003) which implies that patients have to inject relative large amounts of the product, 90 mg subcutaneous, twice daily in order to follow the recommended treatment procedures. It is expected that the T-20 peptide is degraded via hydrolysis by peptidases and proteinases present in various tissues. Thereby, T-20 is degraded in the body to its constituent amino acids with subsequent recycling of the amino acids in the body pool.

The most common adverse events associated with T-20 use are local injection site reactions which are encountered by almost all people receiving T-20. Reactions in the skin where T-20 is injected include itching, swelling, redness, pain or tenderness (Fuzeon label, 2003). Other reported adverse effects of T-20 include discomfort, induration, erythema, nodules and cysts, pruritus, and ecchymosis (Russell et al., 2003).

A further technical problem related to T-20 is the formulation of the final drug. It is generally known that injectable drug formulations shall be as bland and as close as possible to physiological pH and osmolality. Deviation from physiological pH (i.e. a pH around 7.4) and osmolality may result in the patient suffering injection site pain during and after administration of the drug.

The final T-20 drug product (Fuzeon or enfuvirtide) for injection is provided in vials in the form of a white, sterile, lyophilized powder. Prior to subcutaneous administration of Fuzeon, the contents of the vial are reconstituted with sterile water. The reconstituted solution contains approximately 90 mg of T-20 and excipients such as mannitol, sodium carbonate, sodium hydroxide and hydrochloric acid. However, the reconstituted solution has pH of around 9.0, i.e. a pH which deviates significantly from the physiological pH found in humans. The reason may be that the T-20 peptide is found to be most stable and bioactive at pH 9.0. However, as mentioned above, this pH value is not the optimal for injectable drug formulations due to the above mentioned drawbacks.

A further problem is the rapid development of HIV escape mutants and resistance to treatment which has been observed both in vitro with several of the synthetic HR2 derived peptides and the 5-helix bundle fusion protein and in vivo in humans in the clinical trials when T-20 has been applied in monotherapy also in the highest dose. Attempts to solve this rapid development of resistance by the synthesis of the longer HR2 derived peptides T-1249 has appeared promising, but clinical development has recently been halted for this compound. However, increasing the peptide length did slow resistance development in vitro (LEVIN, Jules, 2004; KILBY et al., 2003).

Also, medication based on T-20 is expensive. In the United States, the wholesale acquisition cost for a year's supply of Fuzeon will be around 20,000 USD, and the price in Europe is expected to be similar (STEINBROOK, 2003). The reason for this high cost is primarily due to the fact that T-20 is produced in a highly complicated 106 step solid phase chemical synthesis process (BRAY, 2003). Generally, chemical synthesis of peptides of 30 amino acids or more is difficult and lead even in the best situation to a mixture of compounds with different stereospecificities. Although T-20 has been highly purified unidentified racemic variants are still present in the product and may well influence the production process and quality control from batch to batch.

However, it has now been found by the present inventor, that the above technical problems may be overcome by multimerising (oligomerising) the entire HR2 region or fragments or derivatives of the HR2 region.

The manufacturing the HR2 derivatives, including T-20, through heterologous expression in E. coli or other prokaryotes as multimeric complexes (e.g. trimeric complexes) through genetic fusion of nucleotide sequences encoding the HR2 derivative, with nucleotide sequences encoding a multimerisation domain, allows for the manufacturing of HR2 derivatives of any useful length. Thereby there are provided HR2 based fusion inhibitors having a higher efficacy in preventing the evolution of escape mutants, fusion inhibitors with an increased plasma half-life and increased product uniformity and purity.

It has previously been found the synthetic HR2 derivative T-1249 (which comprises 42 amino acid residues of the HR2 region) in preclinical and clinical analysis has a higher resistance to the evolution of escape mutants. The reasoning behind this is that a full HR2 region derivative will in general, no matter whether the mechanism of escape resides in improved ability to displace the fusion inhibitor compound in six-helix bundle formation or in impairment of accessibility of the HR1 region, be more resistant to viable escape mutant formation compared to a shorter HR2 fragment like T-20. Therefore, the multimerised HIV fusion inhibitors according to the invention may have an increased resistance to viable escape mutant formation as compared to non-multimerised HR2 fragments, because of the avidity gain in effective affinity towards the HR1 region.

T-20's relatively short plasma half life is, as mentioned above, believed to be due to the rapid catabolism of the compound in the liver and in other tissues resulting in a plasma half-life which is too short to provide for suitable administration schedules, as patients have to inject relatively large amounts of the product twice daily. However, by the multimerised HR2 derivatives according to the invention, there is now provided HIV fusion inhibitors which have an improved plasma-half life as compared to presently known fusion inhibitors such as T-20. The increased plasma-half lives that are obtained with the multimerised HR2 derivatives is presently believed to i.a. be due to a higher affinity for serum albumin and other plasma proteins and significant impairment of tissue degradation of the multimerised HR-2 derivatives.

Finally, there is also provided a high yield and simple biological production method and processing process of multimerised HR2 derivatives that results in a single molecular product which can be essentially completely purified from other biological impurities and formulated at physiological pH and osmolarity.

### DISCLOSURE OF THE INVENTION

The primary objective of the present invention is to provide fusion proteins exhibiting antiviral activity, in particular against Human Immunodeficiency Virus (HIV).

More specifically there is provided a fusion protein, capable of forming a multimeric polypeptide complex, exhibiting anti-viral activity which comprises a first polypeptide representing the HR2 region of the ectodomain of the human immunodeficiency virus gp41 protein or a part thereof, and a second polypeptide representing a multimerisation domain peptide.

As mentioned above, the fusion proteins according to the invention, and thereby the multimeric polypeptide complexes formed by these fusion proteins, have several advantageous characteristics as compared to the presently known HIV fusion inhibitors based on the HR2 region of the HIV gp41 protein.

One of these characteristics is that the plasma half-life (also referred to herein as “elimination half-life”) of the HR2 multimers according to the invention is preferably increased as compared to that of a polypeptide comprising the HR2 region of the ectodomain of the HIV gp41 or part thereof, which is not fused or linked to a multimerisation domain. Preferably, the plasma half-life is increased by at least 5%, such as at least 10%, for example at least 15%, such as at least 20%, for example at least 25%, such as at least 30%, for example at least 40% such as at least 50%, for example at least 75%, such as at least 100%. In even more preferred embodiments, the plasma half life is increased at least about 3, 4, 5, 6, 7, 8, 9 or 10 times. In even more preferred embodiments the, plasma half-life is increased at least about 20, 30, 40 or 50 times.

Preferably, the plasma half-life is increased by at least 5%, such as at least 10%, for example at least 15%, such as at least 20%, for example at least 25%, such as at least 30%, for example at least 40% such as at least 50%, for example at least 75%, such as at least 100%. In even more preferred embodiments, the plasma half life is increased at least about 3, 4, 5, 6, 7, 8, 9 or 10 times. In even more preferred embodiments the, plasma half-life is increased at least about 20, 30, 40 or 50 times.

An increased plasma half-life may have profound implications for the use of the multimeric HR2 fusion inhibitors according to the invention in the treatment of HIV infection. It is therefore expected that the clinical effect of the HR2 multimers is superior to the effect of non-multimerised HR2 fragments or derivatives.

Another advantageous characteristic of the HIV fusion inhibitors according to the invention is that their bioavailability is preferably increased as compared to that of a polypeptide comprising the HR2 region of the ectodomain of the HIV gp41 or part thereof, which is not fused or linked to a multimerisation domain. The bioavailability is typically determined by measuring the “Area Under the Curve” (AUC) parameter which is a reflection of the extent of drug bioavailability and graphically consists of the area under the plasma concentration versus time curve. AUC is used extensively in the calculation of drug product performance as this parameter represents the exposure of the patient to the drug after each dose. AUC is usually obtained by a numerical integration procedure known as the trapezoidal rule method e.g. using computer software such as WinNolin, Pharsight Corporation.

In presently preferred embodiments the fusion proteins according to the invention, and thereby the multimeric fusion protein complexes formed thereby, have an AUC value which is increased as compared to that of a polypeptide comprising the HR2 region of the ectodomain of the HIV gp41 or part thereof, which is not fused or linked to a multimerisation domain. Preferably, the AUC value is increased by at least 5%, such as at least 10%, for example at least 15%, such as at least 20%, for example at least 25%, such as at least 30%, for example at least 40% such as at least 50%, for example at least 75%, such as at least 100%. In even more preferred embodiments, the AUC value is increased at least about 3, 4, 5, 6, 7, 8, 9 or 10 times. In even more preferred embodiments the AUC value is increased at least about 20, 30, 40 or 50 times.

In accordance with the invention, the HR2 region of the HIV gp41 protein is linked to a multimerisation domain peptide. In the present context, the term “multimerisation domain” is a peptide, a protein or part of a protein which is capable of interacting with other, similar or identical multimerisation domains. The interaction is of the type that produces multimeric proteins or polypeptides. Such an interaction may be caused by covalent bonds between the components of the multimerisation domains as well as by hydrogen bond forces, hydrophobic forces, van der Waals forces and salt bridges. In useful embodiments, the multimerisation domain peptide is a dimerising domain, a trimerising domain, a tetramerising domain, a pentamerising domain or a hexamerising domain, i.e. domains which are capable of forming polypeptide complexes with two, three, four, five or six HR2 polypeptide entities, respectively.

One example of a multimerisation domain peptide is disclosed in WO 9531540, which describes polypeptides comprising a collectin neck region. The amino acid sequence constituting the collectin neck region may be attached to any polypeptide of choice. Trimers can then be made under appropriate conditions with three polypeptides comprising the collectin neck region amino acid sequence. In further embodiments, the multimerisation domain of the fusion protein according to the invention may comprise coiled-coil dimerization domains such as leucine zipper domains which are found in certain DNA-binding polypeptides.

Advantageously, the multimerisation domain according to the invention may also comprise a dimerization domain which is an immunoglobulin Fab constant domain, such as an immunoglobulin heavy chain CH1 constant region or an immunoglobulin light chain constant region.

In a presently preferred embodiment, the multimerisation domain is derived from tetranectin, and more specifically comprises the tetranectin trimerising structural element (hereafter termed TTSE) which is described in detail in WO 9856906. The amino acid sequence of the TTSE is shown in SEQ ID NO 2. The trimerising effect of TTSE is caused by a coiled coil structure which interacts with the coiled coil structure of two other TTSEs to form a triple alpha helical coiled coil trimer which is exceptionally stable even at relatively high temperatures. The term TTSE is also intended to embrace variants of a TTSE of a naturally occurring member of the tetranectin family of proteins, variants which have been modified in the amino acid sequence without adversely affecting, to any substantial degree, the capability of the TTSE to form alpha helical coiled coil trimers. Thus, the fusion protein according to the invention may comprise a TTSE as a multimerisation domain, which comprises a sequence having at least 68% amino acid sequence identity with the sequence of SEQ ID NO 2, such as at least 75%, including at least 87%, such as at least 92%. In accordance herewith, the cystein residue No. 50 of the TTSE (SEQ ID NO 2) may advantageously be mutagenised to serine, threonine, methionine or to any other amino acid residue in order to avoid formation of an unwanted inter-chain disulphide bridge, which could lead to unwanted multimerisation. TTSE variations may also be obtained by substitution, deletion or insertion of one or more amino acids in the polypeptide representing the TTSE. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 30 amino acids, including the range of 1-20, such as the range of 1-10, including the range of 1-5 amino acids. Obviously, deletions, or truncations, may be performed both at the N-terminus and at the C-terminus of the TTSE. The variation allowed may be determined by systematically making insertions, deletions or substitutions of amino acids in the sequence and testing the resulting variants for their capability to form alpha helical coiled coil trimers.

In a further embodiment, the TTSE multimerisation domain (SEQ ID NO 2) may be modified by (i) the incorporation of polyhistidine sequence and/or a protease cleavage site for e.g. Blood Coagulating Factor Xa or Granzyme B, (ii) replacing Cys 50 with Ser, and (iii) by including a C-terminal KG or KGS sequence. An example of such a modified TTSE is given as SEQ ID NO 3, and is designated TripA.

Useful truncated TTSE variants are shown in the below Table 1:

TABLE 1

| SEQ ID | |
|---|---|
| No. | TTSE variants |
| 50 | EPPTQKPKKIVNAKKDVVNTKMFEELKARLDTLSQEVALLKEQQALQTVSLKG |
| 51 | PPTQKPKKIVNAKKDVVNTKMFEELKARLDTLSQEVALLKEQQALQTVSLKG |
| 52 | PTQKPKKIVNAKKDVVNTKMFEELKARLDTLSQEVALLKEQQALQTVSLKG |
| 53 | TQKPKKIVNAKKDVVNTKMFEELKARLDTLSQEVALLKEQQALQTVSLKG |
| 54 | QKPKKIVNAKKDVVNTKMFEELKARLDTLSQEVALLKEQQALQTVSLKG |
| 55 | KPKKIVNAKKDVVNTKMFEELKARLDTLSQEVALLKEQQALQTVSLKG |
| 56 | PKKIVNAKKDVVNTKMFEELKARLDTLSQEVALLKEQQALQTVSLKG |
| 57 | KKIVNAKKDVVNTKMFEELKARLDTLSQEVALLKEQQALQTVSLKG |
| 58 | KIVNAKKDVVNTKMFEELKARLDTLSQEVALLKEQQALQTVSLKG |
| 59 | IVNAKKDVVNTKMFEELKARLDTLSQEVALLKEQQALQTVSLKG |
| 60 | VNAKKDVVNTKMFEELKARLDTLSQEVALLKEQQALQTVSLKG |
| 61 | NAKKDVVNTKMFEELKARLDTLSQEVALLKEQQALQTVSLKG |
| 62 | AKKDVVNTKMFEELKARLDTLSQEVALLKEQQALQTVSLKG |
| 63 | KKDVVNTKMFEELKARLDTLSQEVALLKEQQALQTVSLKG |
| 64 | KDVVNTKVFEELKARLDTLSQEVALLKEQQALQTVSLKG |
| 65 | VVNTKMFEELKARLDTLSQEVALLKEQQALQTVSLKG |
| 66 | VNTKMFEELKARLDTLSQEVALLKEQQALQTVSLKG |
| 67 | NTKMFEELKARLDTLSQEVALLKEQQALQTVSLKG |
| 68 | TKMFEELKARLDTLSQEVALLKEQQALQTVSLKG |
| 69 | MFEELKARLDTLSQEVALLKEQQALQTVSLKG |
| 70 | EPPTQKPKKIVNAKKDVVNTKMFEELKARLDTLSQEVALLKEQQALQTVSLK |
| 71 | EPPTQKPKKIVNAKKDVVNTKMFEELKARLDTLSQEVALLKEQQALQTVSL |
| 72 | EPPTQKPKKIVNAKKDVVNTKMFEELKARLDTLSQEVALLKEQQALQTVS |
| 73 | EPPTQKPKKIVNAKKDVVNTKMFEELKARLDTLSQEVALLKEQQALQTV |

In accordance with the invention, the polypeptide representing the HR2 region may either be linked to the N- or the C-terminal amino acid residue of the multimerisation domain.

It will be appreciated that a flexible molecular linker optionally may be interposed between, and covalently join, the polypeptide representing the HR2 region and the multimerisation domain. Preferably, the linker is a polypeptide sequence of about 1-20 amino acid residues, such as about 2-10 amino acid residues, including 3-7 amino acid residues. In useful embodiments the linker is essentially non-immunogenic, not prone to proteolytic cleavage and does not comprise amino acid residues which are known to interact with other residues (e.g. cystein residues).

As used herein “a polypeptide representing the HR2 region of the ectodomain of the human immunodeficiency virus gp41 protein” or “the HR2 region” refers to an isolated polypeptide having the amino acid sequence shown in SEQ ID NO 1. Thus, in one embodiment, the fusion protein according to the invention has a first polypeptide representing the HR2 domain which comprises or essentially consists of the amino acid sequence of SEQ ID NO 1. However, it should also be understood that also included in “the HR2 region” definition are parts, fragments, portions and segments of the HR2 region polypeptide (SEQ ID NO 1). Such parts, fragments, portions and segments may be truncated at the N-terminus or C-terminus of the HR2 region, or may lack internal residues, for example, when compared with the full length native HR2 protein. Certain fragments may lack amino acid residues that are not essential for a desired biological activity of the fusion protein according to the invention. Thus, in useful embodiments the fusion protein according to invention may have a first polypeptide representing the HR2 region which comprises or essentially consists of a fragment of the amino acid sequence of SEQ ID NO 1. In presently preferred embodiments the number of amino acids in the fragment is in the range of 20-73 amino acids, such as 30-73 amino acids, including 40-70 amino acids, such as 30-65 amino acids, including 30-60 amino acids, such as 30-55 amino acids, 30-50 amino acids, 30-45 amino acids, 30-40 amino acids and even 30-35 amino acids.

Examples of such useful HR2 region fragments includes or essentially consists of the amino acid sequence of T-20, i.e. amino acid residues 14-64 of SEQ ID NO 1 (SEQ ID NO 4); the amino acid sequence of T1249, i.e. amino acid residues 21-61 of SEQ ID NO 1 (SEQ ID NO 5); the amino acid residues 15-65 of SEQ ID NO 1 (SEQ ID NO 49) representing the HR2 fragment BPFI-0401 shown in the Examples; and SEQ ID NO 159 which is the HR2 fragment used in BPFI-0301 BPFI-0201 and BPFI-0101.

In addition to the full-length HR2 region polypeptides described herein and the parts and fragments thereof, it is contemplated that further multimeric HR2 variants can be prepared. Multimeric HR2 region variants can be prepared by introducing appropriate nucleotide changes into the HR2 DNA. Variations in the full-length HR2 sequence or in various domains of the HR2 region described herein, can be made, for example, using any of the techniques and guidelines for conservative and non-conservative mutations known in the art, for instance, in U.S. Pat. No. 5,364,934. Variations may be a substitution, deletion or insertion of one or more codons encoding the polypeptide representing the HR2 region that results in a change in the amino acid sequence of the polypeptide representing the HR2 region as compared with the native sequence of the HR2 polypeptide. Optionally the variation is by substitution of at least one amino acid with any other amino acid in one or more of the domains of the HR2 region. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 30 amino acids, including the range of 1-20, such as the range of 1-10, including the range of 1-5 amino acids. The variation allowed may be determined by systematically making insertions, deletions or substitutions of amino acids in the sequence and testing the resulting variants for activity exhibited by the full-length HR2 sequence.

Specific examples of useful HR2 variants which may be used in accordance with the invention comprises or essentially consists of an amino acid sequence selected from SEQ ID NO 6, SEQ ID NO 7, SEQ ID NO 8 and SEQ ID NO 9 which represents typical variations in the HR2 regions of various HIV strains.

Specific examples on fragments of the HR2 region which may be used in accordance with the present invention are shown in the below Table 2 (truncations at the C-terminus, SEQ ID Nos. 74-115) and Table 3 (truncations at the N-terminus; SEQ ID Nos. 116-158).

TABLE 2

| | |
|---|---|
| SEQ ID | |
| No. | HR2 fragments-Carboxy truncations |

| | |
|---|---|
| 74 | NASWSNKSLEQIWNNMTWMEWDREINNYTSLIHSLIEESQNQQEKNEQELLELDKWASLWNWFNITNWLWYEK |
| 75 | NASWSNKSLEQTWNNMTWMEWDREINNYTSLIHSLIEESQNQQEKNEQELLELDKWASLWNWFNITNWLWYE |
| 76 | NASWSNKSLEQIWNNMTWMEWDREINNYTSLIHSLIEESQNQQEKNEQELLELDKWASLWNWFNITNWLWY |
| 77 | NASWSNKSLEQIWNNMTWMEWDREINNYTSLIHSLIEESQNQQEKNEQELLELDKWASLWNWFNITNWLW |
| 78 | NASWSNKSLEQIWNNMTWMEWDREINNYTSLIHSLIEESQNQQEKNEQELLELDKWASLWNWFNTTNWL |
| 79 | NASWSNKSLEQIWNNMTWMEWDREINNYTSLIHSLIEESQNQQEKNEQELLELDKWASLWNWFNTTN |
| 80 | NASWSNKSLEQIWNNMTWMEWDREINNYTSLIHSLIEESQNQQEKNEQELLELDKWASLWNWFNIT |
| 81 | NASWSNKSLEQIWNNMTWMEWDREINNYTSLIHSLIEESQNQQEKNEQELLELDKWASLWNWFNI |
| 82 | NASWSNKSLEQIWNNMTWMEWDREINNYTSLIHSLIEESQNQQEKNEQELLELDKWASLWNWFN |
| 83 | NASWSNKSLEQIWNNMTWMEWDREINNYTSLIHSLIEESQNQQEKNEQELLELDKWASLWNWF |
| 84 | NASWSNKSLEQIWNNMTWMEWDREINNYTSLIHSLIEESQNQQEKNEQELLELDKWASLWNW |
| 85 | NASWSNKSLEQIWNNMTWMEWDREINNYTSLIHSLIEESQNQQEKNEQELLELDKWASLWN |
| 86 | NASWSNKSLEQIWNNMTWMEWDREINNYTSLIHSLIEESQNQQEKNEQELLELDKWASLW |
| 87 | NASWSNKSLEQIWNNMTWMEWDREINNYTSLIHSLIEESQNQQEKNEQELLELDKWASL |
| 88 | NASWSNKSLEQIWNNMTWMEWDREINNYTSLIHSLIEESQNQQEKNEQELLELDKWAS |
| 89 | NASWSNKSLEQIWNNMTWMEWDREINNYTSLIHSLIEESQNQQEKNEQELLELDKWA |
| 90 | NASWSNKSLEQIWNNMTWMEWDREINNYTSLIHSLIEESQNQQEKNEQELLELDKW |
| 91 | NASWSNKSLEQIWNNMTWMEWDREINNYTSLIHSLIEESQNQQEKNEQELLELDK |
| 92 | NASWSNKSLEQTWNNMTWMEWDREINNYTSLIHSLIEESQNQQEKNEQELLELD |
| 93 | NASWSNKSLEQIWNNMTWMEWDREINNYTSLIHSLIEESQNQQEKNEQELLEL |
| 94 | NASWSNKSLEQIWNNMTWMEWDREINNYTSLIHSLIEESQNQQEKNEQELLE |
| 95 | NASWSNKSLEQIWNNMTWMEWDREINNYTSLIHSLIEESQNQQEKNEQELL |
| 96 | NASWSNKSLEQIWNNMTWMEWDREINNYTSLIHSLIEESQNQQEKNEQEL |
| 97 | NASWSNKSLEQIWNNMTWMEWDREINNYTSLIHSLIEESQNQQEKNEQE |
| 98 | NASWSNKSLEQIWNNMTWMEWDREINNYTSLIHSLIEESQNQQEKNEQ |
| 99 | NASWSNKSLEQIWNNMTWMEWDREINNYTSLIHSLIEESQNQQEKNE |
| 100 | NASWSNKSLEQIWNNMTWMEWDREINNYTSLIHSLIEESQNQQEKN |
| 101 | NASWSNKSLEQIWNNMTWMEWDREINNYTSLIHSLIEESQNQQEK |
| 102 | NASWSNKSLEQIWNNMTWMEWDREINNYTSLIHSLIEESQNQQE |
| 103 | NASWSNKSLEQIWNNMTWMEWDREINNYTSLIHSLIEESQNQ |
| 104 | NASWSNKSLEQIWNNMTWMEWDREINNYTSLIHSLIEESQN |
| 105 | NASWSNKSLEQIWNNMTWMEWDREINNYTSLIHSLIEESQ |
| 106 | NASWSNKSLEQIWNNMTWMEWDREINNYTSLIHSLIEES |
| 107 | NASWSNKSLEQIWNNMTWMEWDREINNYTSLIHSLIEE |
| 108 | NASWSNKSLEQIWNNMTWMEWDREINNYTSLIHSLIE |
| 109 | NASWSNKSLEQIWNNMTWMEWDREINNYTSLIHSLI |
| 110 | NASWSNKSLEQIWNNMTWMEWDREINNYTSLIHSL |
| 111 | NASWSNKSLEQIWNNMTWMEWDREINNYTSLIHS |
| 112 | NASWSNKSLEQIWNNMTWMEWDREINNYTSLIH |
| 113 | NASWSNKSLEQIWNNMTWMEWDREINNYTSLI |
| 114 | NASWSNKSLEQIWNNMTWMEWDREINNYTSL |
| 115 | NASWSNKSLEQIWNNMTWMEWDREINNYTS |

TABLE 3

| SEQ ID | |
|---|---|
| No. | HR2 fragments-Amino truncations |
| 116 | NASWSNKSLEQIWNNMTWMEWDREINNYTSLIHSLIEESQNQQEKNEQELLELDKWASLWNWFNITNWLWYEK |
| 117 | ASWSNKSLEQIWNNMTWMEWDREINNYTSLIHSLIEESQNQQEKNEQELLELDKWASLWNWFNITNWLWYEK |
| 118 | SWSNKSLEQIWNNMTWMEWDREINNYTSLIHSLIEESQNQQEKNEQELLELDKWASLWNWFNITNWLWYEK |
| 119 | SNKSLEQIWNNMTWMEWDREINNYTSLIHSLIEESQNQQEKNEQELLELDKWASLWNWFNITNWLWYEK |
| 120 | NKSLEQIWNNMTWMEWDREINNYTSLIHSLIEESQNQQEKNEQELLELDKWASLWNWFNITNWLWYEK |
| 121 | KSLEQIWNNMTWMEWDREINNYTSLIHSLIEESQNQQEKNEQELLELDKWASLWNWFNITNWLWYEK |
| 122 | SLEQIWNNMTWMEWDREINNYTSLIHSLIEESQNQQEKNEQELLELDKWASLWNWFNITNWLWYEK |
| 123 | LEQIWNNMTWMEWDREINNYTSLIHSLIEESQNQQEKNEQELLELDKWASLWNWFNITNWLWYEK |
| 124 | EQIWNNMTWMEWDREINNYTSLIHSLIEESQNQQEKNEQELLELDKWASLWNWFNITNWLWYEK |
| 125 | QIWNNMTWMEWDREINNYTSLIHSLIEESQNQQEKNEQELLELDKWASLWNWFNITNWLWYEK |
| 126 | IWNNMTWMEWDREINNYTSLIHSLIEESQNQQEKNEQELLELDKWASLWNWFNITNWLWYEK |
| 127 | WNNMTWMEWDREINNYTSLIHSLIEESQNQQEKNEQELLELDKWASLWNWFNITNWLWYEK |
| 128 | NNMTWMEWDREINNYTSLIHSLIEESQNQQEKNEQELLELDKWASLWNWFNITNWLWYEK |
| 129 | NMTWMEWDREINNYTSLIHSLIEESQNQQEKNEQELLELDKWASLWNWFNITNWLWYEK |
| 130 | MTWMEWDREINNYTSLIHSLIEESQNQQEKNEQELLELDKWASLWNWFNITNWLWYEK |
| 131 | TWMEWDREINNYTSLIHSLIEESQNQQEKNEQELLELDKWASLWNWFNITNWLWYEK |
| 132 | WMEWDREINNYTSLIHSLIEESQNQQEKNEQELLELDKWASLWNWFNITNWLWYEK |
| 133 | MEWDREINNYTSLIHSLIEESQNQQEKNEQELLELDKWASLWNWFNITNWLWYEK |
| 134 | EWDREINNYTSLIHSLIEESQNQQEKNEQELLELDKWASLWNWFNITNWLWYEK |
| 135 | WDREINNYTSLIHSLIEESQNQQEKNEQELLELDKWASLWNWFNITNWLWYEK |
| 136 | DREINNYTSLIHSLIEESQNQQEKNEQELLELDKWASLWNWFNITNWLWYEK |
| 137 | REINNYTSLITHSLIEESQNQQEKNEQELLELDKWASLWNWFNITNWLWYEK |
| 138 | EINNYTSLIHSLIEESQNQQEKNEQELLELDKWASLWNWFNITNWLWYEK |
| 139 | INNYTSLIHSLIEESQNQQEKNEQELLELDKWASLWNWFNITNWLWYEK |
| 140 | NNYTSLIHSLIEESQNQQEKNEQELLELDKWASLWNWFNITNWLWYEK |
| 141 | NYTSLIHSLIEESQNQQEKNEQELLELDKWASLWNWFNITNWLWYEK |
| 142 | YTSLIHSLIEESQNQQEKNEQELLELDKWASLWNWFNITNWLWYEK |
| 143 | TSLIHSLIEESQNQQEKNEQELLELDKWASLWNWFNITNWLWYEK |
| 144 | SLIHSLIEESQNQQEKNEQELLELDKWASLWNWFNITNWLWYEK |
| 145 | LIHSLIEESQNQQEKNEQELLELDKWASLWNWFNITNWLWYEK |
| 146 | IHSLIEESQNQQEKNEQELLELDKWASLWNWFNITNWLWYEK |
| 147 | HSLIEESQNQQEKNEQELLELDKWASLWNWFNITNWLWYEK |
| 148 | SLIEESQNQQEKNEQELLELDKWASLWNWFNITNWLWYEK |
| 149 | LIEESQNQQEKNEQELLELDKWASLWNVFNITNWLWYEK |
| 150 | IEESQNQQEKNEQELLELDKWASLWNWFNITNWLWYEK |
| 151 | EESQNQQEKNEQELLELDKWASLWNWFNITNWLWYEK |
| 152 | ESQNQQEKNEQELLELDKWASLWNWENITNWLWYEK |
| 153 | SQNQQEKNEQELLELDKWASLWNWFNITNWLWYEK |
| 154 | QNQQEKNEQELLELDKWASLWNWFNITNWLWYEK |
| 155 | NQQEKNEQELLELDKWASLWNWFNITNWLWYEK |
| 156 | QQEKNEQELLELDKWASLWNWFNITNWLWYEK |
| 157 | QEKNEQELLELDKWASLWNWFNITNWLWYEK |
| 158 | EKNEQELLELDKWASLWNWFNITNWLWYEK |

Preferably, the term “HIV” as used herein refers to HIV-1. However it is to be understood that while HIV viral infection are being used herein as a model system in which the potential anti-viral activity of the multimeric fusion protein according to the invention are described, such anti-viral activity may represent a generalised mechanism by which a broad spectrum of enveloped viral infections may be inhibited. Enveloped viruses whose infectivity may be inhibited using the multimeric fusion protein of the invention may include, but are not limited to, other HIV strains such as HIV-2.

In one useful embodiment, the fusion protein according to the invention is selected from BPFI-0100 (SEQ ID NO 10), BPFI-0200 (SEQ ID NO 11), BPFI-0300 (SEQ ID NO 12), BPFI-0101 (SEQ ID NO 42), BPFI-0201 (SEQ ID NO 43) and BPFI-0301 (SEQ ID NO 44).

As will be apparent from the following Examples, the trimeric fusion proteins BPFI-0100 (SEQ ID NO 10), BPFI-0200 (SEQ ID NO 11) and BPFI-0300 (SEQ ID NO 12) all exhibited an antiviral activity (IC50, 50% inhibitory concentration) which was similar or slightly better as compared to the T-20 compound and the monomeric HR2 fragment BPFI-0400.

As will also be apparent from the Examples, the trimeric fusion proteins BPFI-0101 (SEQ ID NO 42), BPFI-0201 (SEQ ID NO 43) and BPFI-0301 (SEQ ID NO 44) showed particularly good characteristics with respect to elimination half-life (plasma half-life) and bioavailability. It was demonstrated that the trimerised HR2 fragments (BPFI-0101, BPFI-0201, BPFI-0301) all have an elimination half-life which is significantly longer than the monomeric HR2 fragments T-20 and BPFI-0401. Thus, it was found that BPFI-0101 has an elimination half-life of 12 hours; BPFI-0201 an elimination half-life of 10 hours and BPFI-0301 an elimination half-life of 9 hours. In contrast hereto, the monomeric HR2 fragments BPFI-0401 and T-20 both had an elimination half-life of 2 hours.

It was also found that the trimerised HR2 fragments BPFI-0101, BPFI-0201 and BPFI-0301 all have an Area Under the Curve value (AUC) which is higher than for T-20. It was surprisingly found that the trimeric HR2 fragment BPFI-0301 has an AUC, and thereby a bioavailability, which is 3.5 times higher than T-20 and the monomeric BPFI-0401. BPFI-0301 is trimerised using the 37 amino acid trimerisation module (TTSE) fragment (V17; SEQ ID NO 65) which was the shortest of the applied TTSE's. The TTSE's applied in BPFI-0101 and BPFI-0201 contained 53 amino acids (E1; SEQ ID NO 50) and 44 amino acids (110; SEQ ID NO 59), respectively. This suggests that it may be factors other than the actual size of the fusion protein, and thereby the size of the trimeric complex, which influence the AUC.

One important aspect is that the fusion proteins according to invention, and thereby the multimeric fusion proteins formed thereby, may be formulated at physiological pH. As mentioned above, it is important that injectable drug formulations are as close as possible to physiological pH and osmolality, as deviation from physiological pH (i.e. a pH around 7.4) and osmolality may result in adverse injection site reactions. As can be seen from Example 5, this was demonstrated by readily dissolving lyophilised trimeric BPFI-0301 into a buffer containing 20 mM NaHCO₃ at pH 7.4 at a concentration of 110 mg/mL.

Preferably, the fusion proteins according to the invention are stable, bioactive and may be formulated at a pH in the range of 7.00-8.00, such as in the in the range of 7.10-7.20, in the range of 7.20-7.30, in the range of 7.30-7.40, in the range of 7.40-7.50, in the range of 7.50-7.60, in the range of 7.60-7.70, in the range of 7.70-7.80, in the range of 7.80-7.90 or in the range of 7.90-8.00.

The fusion proteins of the present invention may be chemically synthesised or expressed in any suitable standard protein expression system. Preferably, the protein expression systems are systems from which the desired protein may readily be isolated and refolded in vitro. As a general matter, prokaryotic expression systems are preferred since high yields of protein can be obtained and efficient purification and refolding strategies are available. Thus, it is well within the abilities and discretion of the skilled artisan to choose an appropriate or favourite expression system. Similarly, once the primary amino acid sequence for the fusion proteins of the present invention is chosen, one of ordinary skill in the art can easily design appropriate recombinant DNA constructs which will encode the desired proteins, taking into consideration such factors as codon biases in the chosen host, the need for secretion signal sequences in the host, the introduction of proteinase cleavage sites within the signal sequence, and the like. These recombinant DNA constructs may be inserted in-frame into any of a number of expression vectors appropriate to the chosen host. Preferably, the expression vector will include a strong promoter to drive expression of the recombinant constructs.

In a presently preferred production method, the fusion protein of the invention, comprising a first polypeptide (HR2 region) and a second polypeptide (multimerisation domain) is expressed in a prokaryotic host cell such as E. coli and is additionally linked to a third polypeptide, i.e. a third fusion partner. Thus, it has surprisingly been found, that by adding such third fusion partner to the fusion protein of the invention, high yields of the fusion protein may be obtained. The third fusion partner may, in accordance with the invention, be of any suitable kind provided that it is a peptide, oligopeptide, polypeptide or protein, including a di-peptide, a tri-peptide, a tetra-peptide, penta-peptide and a hexa-peptide. The fusion partner may in certain instances be a single amino acid. It may be selected such that it renders the fusion protein more resistant to proteolytic degradation, facilitate enhanced expression and secretion of the fusion protein, improve solubility, and/or allow for subsequent affinity purification of the fusion protein. In a presently preferred embodiment, the third fusion partner is the polypeptide ubiquitin.

Preferably, the junction region between the fusion protein of the invention (i.e. the first polypeptide representing the HR2 region and the second polypeptide representing the multimerisation domain) and the third fusion partner such as ubiquitin, comprises a Granzyme B protease cleavage site such as human Granzyme B (E.C. 3.4.21.79). More detailed information on the use of Granzyme B as fusion protein cleaving agent may be found in PCT Application No. WO 2004094478.

The third fusion partner may in further useful embodiments be coupled to an affinity-tag. Such an affinity-tag may e.g. be an affinity domain which permits the purification of the fusion protein on an affinity resin. The affinity-tag may be a polyhistidine-tag including hexahis-tag, a polyarginine-tag, a FLAG-tag, a Strep-tag, a c-myc-tag, a S-tag, a calmodulin-binding peptide, a cellulose-binding peptide, a chitin-binding domain, a glutathione S-transferase-tag, or a maltose binding protein.

The method according to the invention may in useful embodiments include an isolation step for isolating the fusion protein of the invention which is formed by the enzymatic cleavage of the fusion protein, which has e.g. been immobilised by the use of the above mentioned affinity-tag systems. This isolation step can be performed by any suitable means known in the art for protein isolation, including the use of ion exchange and fractionation by size, the choice of which depends on the character of the fusion protein. In on presently preferred embodiment, the region between the third fusion partner and the region comprising the first polypeptide (HR2 region) and the second polypeptide (multimerisation domain) is contacted with the human serine protease Granzyme B to cleave of the fusion protein at a Granzyme B protease cleavage site to yield the fusion protein of the invention.

When one or more polypeptides representing the HR2 region is coupled to an multimerisation domain, and thereby forming the fusion protein according to the invention, multimers of the HR2 region can be made by contacting the fusion proteins under appropriate conditions resulting in a multimeric polypeptide complex. In this way HR2 region dimers, trimers, tretramers, pentamers, hexamers or even higher-mers can be prepared depending on the type of multimerisation domain being linked to the HR2 region. Thus, in one aspect of the invention there is provided a multimeric polypeptide complex comprising at least two fusion proteins, such as at least three, including at least four, such at least five, including at least six fusion proteins. The presence of a HR2 region multimer, such as a HR2 region trimer, may be ascertained by well known techniques such as gelfiltration, SDS-PAGE, or native SDS gel electrophoresis depending on the nature of the multimer.

In one aspect the polypeptide complex comprises three fusion proteins trimerised e.g. by the use of the tetranectin trimerising structural element.

The anti-viral activity exhibited by the multimeric fusion proteins of the invention, may be measured by suitable assays well-known in the art, for example by contacting to an HIV-infected cell an effective fusion inhibiting amount of the multimeric protein according to the invention. The assay may be carried out as an in vitro assay, e.g. as the one described in Example 4 below, using strain IIIB HIV-1 infected human T cells (lymphoblast cell line MT4). The assay may also be carried out in vivo in an animal subject infected with the HIV-virus. In a presently preferred embodiment, the polypeptide complex according to invention exhibits an in-vitro antiviral activity against strain IIIB of HIV-1 using MT4 cells as target cells, with an 50% inhibitory concentration (IC50) in the range of 1-500 nM, such as in the range of 1-400 nM, 1-300 nM, 1-200 nM, 1-100 nM, 1-50 nM, 1-40 nM, 1-30 nM, 1-20 nM, and in the range of 1-10 nM.

The fusion protein according to the invention may be used for the preparation of a pharmaceutical composition by any suitable method well known in the art. The composition may together with the multimeric HR2 region fusion protein, comprise one or more acceptable carriers therefore, and optionally other therapeutic ingredients. The carriers must be acceptable in the sense of being compatible with the other ingredients and not deleterious to the recipient thereof. In general, methods for the preparation of pharmaceutical compositions include the step of bringing into association the active ingredient and a carrier.

The therapeutic application of the polypeptides of the present invention comprises use of the polypeptides as inhibitors of human and non-human retroviral transmission to uninfected cells. The human retroviruses that may be inhibited by the fusion proteins of the invention include all strains of HIV-1 and HIV-2.

Thus, the polypeptide complex of the invention may used as a therapeutic in the treatment of AIDS by administering a therapeutically effective amount of the polypeptide complex according to the invention to a subject in need thereof.

The polypeptide complex of the invention may be administered directly to the subject by any suitable technique, including parenterally, and can be administered locally or systemically. The specific route of administration depends, e.g., on the medical history of the subject. Examples of parenteral administration include subcutaneous, intramuscular, intravenous, intraarterial, and intraperitoneal administration. For injection, the proteins of the invention may be formulated in aqueous solutions, preferably in physiologically compatible buffers such as Hanks' solution, Ringer's solution, or physiological saline buffer.

Optionally, the administration of the polypeptide complex may comprise the administration of at least one further therapeutic agent, such as other antiretroviral agents, including protease inhibitors, non-nucleoside reverse transcriptase inhibitors, and nucleoside/nucleotide reverse transcriptase inhibitors.

The fusion protein or the polypeptide complex according to the invention may be used for the preparation of a pharmaceutical composition by any suitable method well known in the art. The composition may together with the multimeric HR2 fusion protein, comprise one or more acceptable carriers therefore, and optionally other therapeutic ingredients. The carriers must be acceptable in the sense of being compatible with the other ingredients and not deleterious to the recipient thereof. In general, methods for the preparation of pharmaceutical compositions include the step of bringing into association the active ingredient and a carrier.

The invention will now be described by way of illustration in the following non-limiting examples and drawings.

### BRIEF DESCRIPTION OF THE DRAWINGS

FIG. 1: Schematic representation of the HIV gp41 ectodomain. The HIV gp41 ectodoamin is comprised by the following functional and structural regions (in the sequential order they appear from the amino terminus): Fusion peptide (FP), which is inserted into the target cell membrane, the helix forming region one (HR1), which forms the central trimeric coiled coil structure in the six-helix bundle structure, the cystein bridge (cys bridge) containing bridging region, the second helix forming region (HR2), which represent the outer anti-parallel strands in the six-helix bundle structure, followed by the transmembrane region (TM).

FIG. 2: Schematic outline of the fusion proteins UB-BPFI-0100, -0200, -0300, -0400 as well as the protease processed fusion proteins BPFI-0100, -0200, -0300, and -0400. The fusion proteins UB-BPFI-0100, -0200, and -0300 were constructed from a common general structure. These fusion proteins are comprised by the following functional and structural regions (in the sequential order they appear from the amino terminus): The six histidine tag (H6), which confers affinity of the fusion protein towards the Ni-NTA matrix, human ubiquitin (UB), the granzyme B cleavage site signal (GrB), the HIV gp41 helix region 2 (HR2), one of the three trimerisation domains E1-Trip, I10-Trip, and V17-Trip, followed by the myc tag. The fusion protein UB-BPFI-0400 does not contain a timerisation domain and it contains an e-tag instead of the myc tag.

FIG. 3: SDS-PAGE analysis of the expressed and purified UB-BPFI fusion proteins, the cleavage products and the purified BPFI fusion protein. A: Lane M, marker proteins (from top to bottom) 94 kDa, 67 kDa, 43 kDa, 30 kDa, 20 kDa, and 14.4 kDa. Lane 1: UB-BPFI-0100, lane 2: GrB cleavage of UB-BPFI-0100, lane 3: UB-BPFI-0200, lane 4: GrB cleavage of UB-BPFI-0200, lane 5: UB-BPFI-0300, lane 6: GrB cleavage of UB-BPFI-0300, lane 7: UB-BPFI-0400, and lane 8: GrB cleavage of UB-BPFI-0400. B: Lane M, marker proteins (from top to bottom) 94 kDa, 67 kDa, 43 kDa, 30 kDa, 20 kDa, and 14.4 kDa. Lane 1: BPFI-0400, lane 2: BPFI-0300, lane 3: BPFI-0200, and lane 4: BPFI-0100.

FIG. 4: Results of BPFI-0300 inhibition analysis of HIV-1 strain IIIB infection of MT4 cells.

FIG. 5: Schematic representation of the amino acid sequences for T20, BPFI-0401, BPFI-0301, BPFI-0201 and BPFI-0101. Amino acids in bold indicates the tetranectin trimerisation unit derivatives: V17-Trip was applied in BPFI-0301, 110-Trip was applied in BPFI-0201 and E1-Trip was applied in BPFI-0101. Dotted lines indicate amino acids which are not shown.

FIG. 6: Clearing curve from a toxicokinetic analysis in rats subcutaneously injected with a 5 mg/kg dose of radio-labelled BPFI-0301 at time zero.

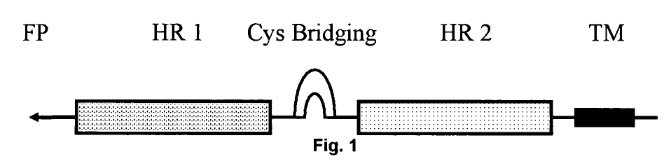

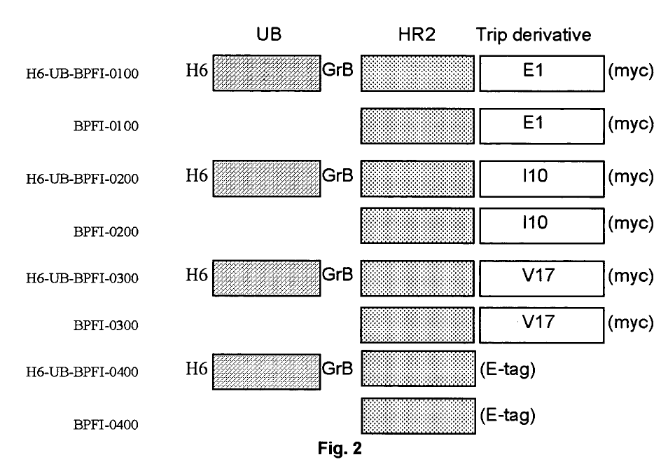

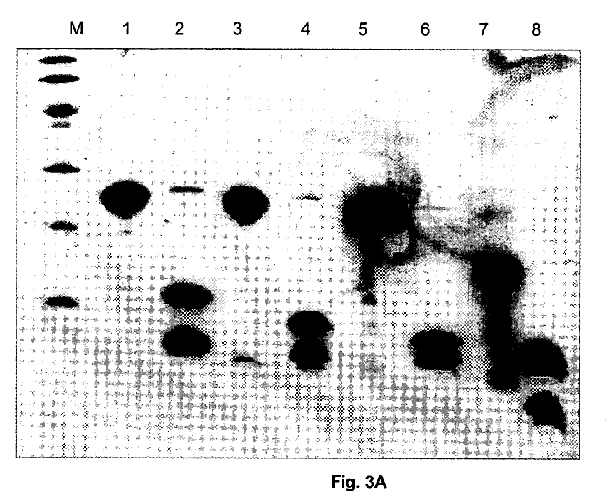

### EXAMPLE 1

Construction of pT7H6UB-BPFI Expression Vectors 0100, 0200, 0300 and 0400

A DNA fragment encoding the HIV gp41 HR2 domain, SEQ ID NO 14, was synthesised in a 50 μL assembly reaction of 10 pmole of the synthetic oligonucleotides 5hr23, SEQ ID NO 15, and 3hr25, SEQ ID NO 16, over 10 cycles using the thermostable DNA polymerase Immolase, 0.2 mM of each of the deoxynucleotide triphophates dATP, dTTp, dCTP, and dGTP in a suitable buffer supplied by the manufacturer. The temperature-time program of each cycle was: 95° C.—30 secs, 54° C.—60 secs, and 72° C.—60 secs on a DYAD DNA engine thermocycler. The cycle program was initiated with a 5 min treatment of the reaction mixture at 95° C. All synthetic oligonucleotides used in this example were provided by DNA Technology (Aarhus, Denmark). After completion of the assembly reaction 5 μL of the reaction solution was used as template in a second 50 μL PCR reaction over 15 cycles aiming at trimming the HR2 domain with appropriate restriction nuclease sites for cloning using 20 pmoles of each of the synthetic oligonucleotides nest-5hr23, SEQ ID NO 17, and nest-3hr25, SEQ ID NO 18, the thermostable DNA polymerase Immolase, 0.2 mM of each of the deoxynucleotide triphophates dATP, dTTp, dCTP, and dGTP in a suitable buffer supplied by the manufacturer. The temperature-time program was initiated with a so-called hot-start step at 95° C. for 5 min followed by 15 cycles where each cycle was: 95° C.—30 secs, 54° C.—60 secs, and 72° C.—60 secs on a DYAD DNA engine thermocycler. The resulting DNA fragment was isolated from the PCR reaction mixture by phenol/chloroform extraction and ethanol precipitation. The dried DNA pellet was dissolved in water. Five μL of the solution was digested in two successive 50 μL reactions with the restriction endonucleases Bam HI and Kpn I (New England Biolabs) at 37° C. for two hours. The HR2 encoding fragment was cloned into the three E. coli plasmids pT7-Tripmyc, -I10Tripmyc, and -V17Tripmyc after restriction endonuclease cleavage yielding the three plasmids pT7-HR2-Tripmyc (SEQ ID NO 19), pT7-HR2-I10Tripmyc (SEQ ID NO 20), pT7-HR2-V17Tripmyc (SEQ ID NO 21), respectively. The nucleotide sequence of the HR2 inserts in the three plasmids was confirmed by nucleotide sequencing using standard procedures. Each of the three plasmids pBAD-HR2-Trip, pBAD-HR2-I10Trip, and pBAD-HR2-V17Trip were used in three PCR-reactions aiming at amplifying the HIV gp41 HR2 domain fused to full length, 110 and V17 derivatives of the trimerisation unit from the human protein Tetranectin (SEQ ID NO 2) together with the so-called myc tag for subcloning into the E. coli fusion protein expression vector pT7H6-UB. Twenty pmole of each synthetic oligonucleotide nest-5hr23-2, SEQ ID NO 22 and myc-C, SEQ ID NO 23 were used as primers for the PCR-reactions together with the thermostable DNA polymerase Immolase, 0.2 mM of each of the deoxynucleotide triphophates dATP, dTTp, dCTP, and dGTP in a suitable buffer supplied by the manufacturer. The temperature-time program was initiated with a so-called hot-start step at 95° C. for 5 min followed by 30 cycles where each cycle was: 95° C.—30 secs, 54° C.—60 secs, and 72° C.—60 secs on a DYAD DNA engine thermocycler. The resulting DNA fragments were isolated from the PCR reaction mixture by phenol/chloroform extraction and ethanol precipitation. The dried DNA pellets were dissolved in water. Five μL of the solutions were digested in 50 μL reactions with the restriction endonucleases Bam HI and Hind III (New England Biolabs) at 37° C. for two hours. After restriction endonuclease cleavage the HR2-Trip-myc derivative encoding fragments were cloned into the Bam HI and Hind III cleaved E. coli expression plasmid pT7H6-UB. The nest-5hr23-2 primer encodes a recognition and cleavage site IEPD (SEQ ID NO 24) for the human serine protease Granzyme B in the 5′-end of the oligonucleotide. The nucleotide sequence of the fusion proteins encoded by the resulting plasmids pT7H6-UB-BPFI-0100 (SEQ ID NO 25), pT7H6-UB-BPFI-0200 (SEQ ID NO 26), and pT7H6-UB-BPFI-0300 (SEQ ID NO 27), respectively were confirmed by nucleotide sequence analysis using standard procedures, and the amino acid sequence of the encoded fusion proteins H6-UB-BPFI-0100 (SEQ ID NO 28), -0200 (SEQ ID NO 29), and 0300 (SEQ ID NO 30). The amino acid sequence of the processed fusion proteins BPFI-0100, -0200, and -0300 are shown in SEQ ID NO 10, SEQ ID NO 11, and SEQ ID NO 12, respectively.

The pBAD-HR2-Trip plasmid was further used in a PCR-reaction aiming at amplifying the HIV gp41 HR2 domain fused to the so-called E-tag (Amersham Biosciences) for subcloning into the E. coli fusion protein expression vector pT7H6-UB. Twenty pmole of each synthetic oligonucleotide nest-5hr23-2, SEQ ID NO 22 and ET-HR2-3, SEQ ID NO 31 were used as primers for the PCR-reactions together with the thermostable DNA polymerase Immolase, 0.2 mM of each of the deoxynucleotide triphophates dATP, dTTp, dCTP, and dGTP in a suitable buffer supplied by the manufacturer. The temperature-time program was initiated with a so-called hot-start step at 95° C. for 5 min followed by 30 cycles where each cycle was: 95° C.—30 secs, 54° C.—60 secs, and 72° C. —60 secs on a DYAD DNA engine thermocycler. The resulting DNA fragment was isolated from the PCR reaction mixture by phenol/chloroform extraction and ethanol precipitation. The dried DNA pellet was dissolved in water. Five μL of the solution was digested in a 50 μL reaction with the restriction endonucleases Bam HI and Hind III (New England Biolabs) at 37° C. for two hours. After restriction endonuclease cleavage the HR2-E-tag encoding fragment was cloned into the Bam HI and Hind III cleaved E. coli expression plasmid pT7H6-UB. The nest-5hr23-2 primer SEQ ID NO 22 encodes a recognition and cleavage site IEPD, SEQ ID NO 24 for the human Granzyme B seine protease in the 5′-end of the oligonucleotide. The nucleotide sequence of the fusion protein encoded by the resulting plasmid pT7H6-UB-BPFI-0400 (SEQ ID NO 32) was confirmed by nucleotide sequence analysis using standard procedures, and the amino acid sequence of the encoded fusion proteins H6-UB-BPFI-0400 is shown in SEQ ID NO 33, and the amino acid sequence of the processed fusion proteins BPFI-0400 is shown in SEQ ID NO 13.

### EXAMPLE 2

Construction of pT7H6UB-BPFI Expression Vectors 0101, 0201, 0301 and 0401

The E. coli expression vectors pT7H6UB-BPFI-0101, -0201, and -0301 encoding the H6UB-BPFI-HR2 fusion protein fused to the Tetranectin trimerisation module derivatives E1 (SEQ ID NO 50), 110 (SEQ ID NO 59), and V17 (SEQ ID NO 65), respectively, were constructed by site directed mutagenesis of the GGT codon encoding the glycine residue number 196, 187, or 179, respectively in the corresponding fusion proteins H6UB-BPFI-0100, 0200, or -0300 to the translation stop codon TAA using the oligonucleotide primer pair FI-myc-deI-5 SEQ ID NO 34 and FI-myc-deI-3 SEQ ID NO 35 and the Quick Change Mutagenesis kit (Stratagene) as described by the manufacturer. The nucleotide sequence of the H6UB-BPFI-0101, -0201, and -0301 encoding regions were confirmed by nucleotide sequencing (SEQ ID NO: 36, 37 and 38, respectively). The amino acid sequence of the encoded fusion proteins H6-UB-BPFI-0101, -0201, and 0301 are shown in SEQ ID NO 39, SEQ ID NO 40 and SEQ ID NO 41, respectively. The amino acid sequence of the processed fusion proteins BPFI-0101, -0201, and -0301 are shown in SEQ ID NO 42, SEQ ID NO 43 and SEQ ID NO 44, respectively.

The E. coli expression vector pT7H6UB-BPFI-0401 encoding the H6UB-BPF1—HR2 fusion protein was constructed by site directed mutagenesis of the GGT codon encoding the glycine residue number 142 of the H6UB-BPFI-0400 fusion protein to the translation stop codon TAA using the oligonucleotide primer pair FI-ET-deI-5 SEQ ID NO 45 and FI-ET-deI-3 SEQ ID NO 46 and the Quick Change Mutagenesis kit (Stratagene) as described by the manufacturer. The nucleotide sequence of the H6UB-BPFI-0401 encoding region was confirmed by nucleotide sequencing (SEQ ID NO: 47). The amino acid sequence of the encoded fusion proteins H6-UB-BPFI-0401 is shown in SEQ ID NO 48. The amino acid sequence of the processed fusion proteins BPFI-0401 is shown in SEQ ID NO 49.

### EXAMPLE 3

Production, Purification and Processing of HIV gp41 HR2 Derivatives BP-FI-0100, BP-FI-0200, BP-FI-0300, and BP-FI-0400

Construction of the T7 RNA polymerase dependent expression plasmids pT7H6-UB-FI-0100, pT7H6-UB-FI-0200, pT7H6-UB-FI-0300 (expressing trimersed fusion protein derivatives of the HIV gp41 HR2 domain with a C-terminal myc tag), and pT7H6-UB-FI-0400 (expressing monomeric HR2 fusion derivative with a C-terminal E-tag) is described in Example 1.

The fusion proteins H6-UB-FI-0100 to -0400 were produced by growing and expressing each of the expression plasmids pT7H6-UB-FI-0100 to -0400 in E. coli BL21 cells in a medium scale (6×1 litre) as described by STUDIER, et al. Journal of Molecular Biology. 1986, vol. 189, p. 113-130. Briefly, exponentially growing cultures at 37° C. were at OD600 0.8 infected with bacteriophage λ-CE6 at a multiplicity of approximately 5. One hour after infection 0.2 g of rifampicin was added to each litre of culture in order to boost expression level. Cultures were grown at 37° C. for another three hours before cells were harvested by centrifugation. Cells were lysed by osmotic shock and sonification and total cellular protein extracted into phenol (adjusted to pH 8 with Trisma base). Protein was precipitated from the phenol phase by addition of 2.5 volumes of ethanol and centrifugation. The protein pellet was dissolved in a buffer containing 6 M Guanidinium chloride, 50 mM Tris-HCL pH 8 and 50 mM dithio-erythriol. Following gel filtration on Sephadex G-25 (Amersham Biosciences) into 8 M Urea, 0.5 M NaCl, 50 mM Tris-HCL pH 8, and 5 mM 2-mercaptoethanol, the crude protein preparation was applied to Ni2+ activated NTA-agarose (Qiagen, Germany) columns for purification (HOCHULI, et al. Biotechnology. 1988, p. 1321-1325.) of the fusion proteins.

All buffers prepared for liquid chromatography were degassed under vacuum prior to addition of reductant and/or use.

Upon application of the crude protein extracts on the Ni2+ NTA-agarose column, the H6-UB-FI-0100, H6-UB-FI-0200, H6-UB-FI-0300, and H6-UB-FI-0400 fusion proteins, respectively were purified from the majority of coli and λ phage proteins by washing with two column volumes of 6 M Guanidinium chloride, 50 mM Tris-HCL and 5 mM 2-mercaptoethanol buffer, followed by 8 M Urea, 50 mM Tris-HCl pH 8, 0.5M NaCl until the optical density (OD) at 280 nm of the column eluates were stable.

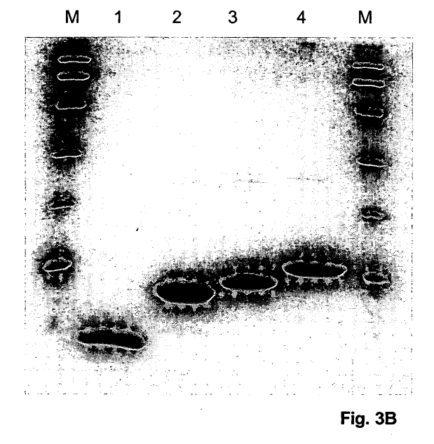

The fusion proteins were eluted from the Ni²⁺NTA-agarose columns with a buffer containing 8 M Urea, 0.5 M NaCl, 50 mM Tris-HCL and 10 mM EDTA pH 8, and refolded by buffer exchange to 10 mM Tris-HCL pH 7.5, 25 mM NaCl on Sephadex G-25 columns (FIG. 3). Yield of purified and refolded H6-UB-BPFI-0100, -0200, and -0300 fusion proteins were between 25 to 30 mg/litre culture, and 50 mg/litre culture of H6-UB-BPFI-0400. Approximately 25 mg of each fusion protein was cleaved for 2 hours at room temperature with recombinant human serine protease Granzyme B in a weight/weight ratio of 2000/1. The cleavages were analysed by SDS-PAGE and found to be near complete (FIG. 3). After cleavage the protein solutions were loaded onto Q-Sepharose (AmershamBiosciences) columns and submitted to ion-exchange chromatography by elution with a liner gradient from 25 mM NaCl, 10 mM Tris-HCL pH 7.5 to 750 mM NaCl, 10 mM Tris-HCL pH 7.5 over 10 column volumes. All fully processed BP-FI fusion proteins eluted around 500 mM NaCl. Pooled fractions of fully processed and pure BP-FI-0100, BP-FI-0200, BP-FI-0300, and BP-FI-0400 were analysed by SD-PAGE (FIG. 3). Process yields were: BPFI-0100 (13 mg/L culture); BPFI-0200 (11 mg/L culture); BPFI (18 mg/L culture); and BPFI-0400 (24 mg/L culture).

### EXAMPLE 4

Head to Head Analysis of the HIV gp41 HR2 Derivatives BPFI-0100, -0200, -0300, and -0400 and T-20 (Fuzeon, Enfuvirtide) Anti-HIV-1 Activity In Vitro

The purified and fully processed trimeric BPFI-0100, BPFI-0200, BPFI-0300, and the monomeric BPFI-0400 fusion proteins, in 10 mM Tris-HCl pH 7.5; 0.5 M NaCl, and the commercially available synthetic peptide T-20 (Fuzeon, Roche) were analysed for their possible antiviral activity against the strain IIIB of HIV-1 using the standard human T cell lymphoblast cell line MT4 (obtained from the European Collection of Cell Cultures, ECACC). HIV-1 expression in the cell cultures were quantified indirectly by the standard MTT cell proliferation assay (R&D Systems, Abingdon, U.K.).

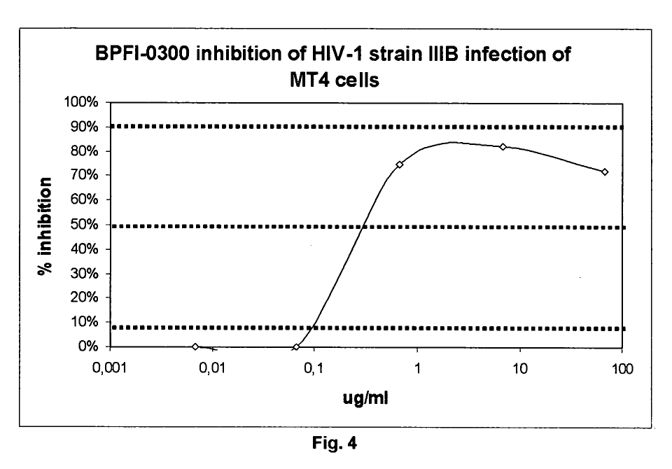

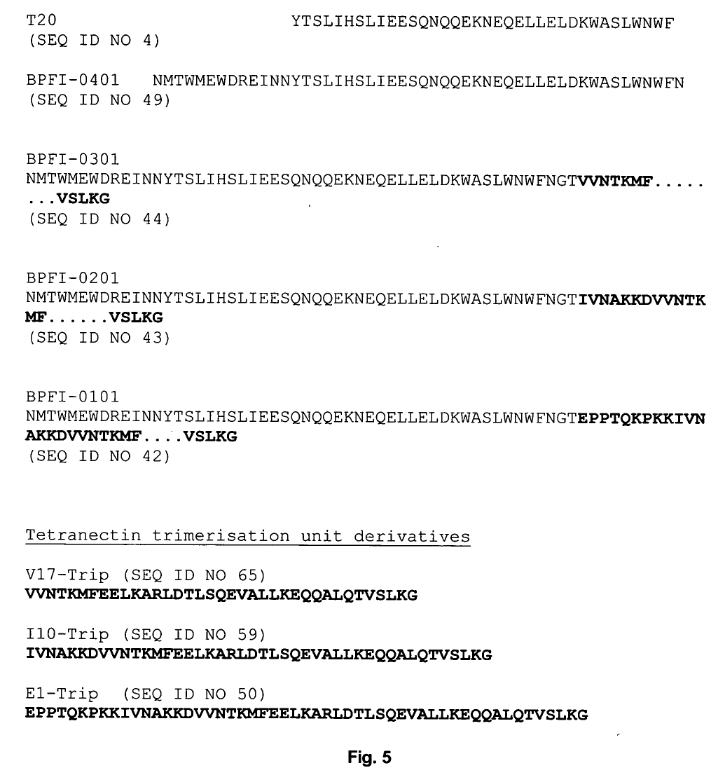

Briefly, HIV-1 strain IIIB (obtained from NIH AIDS Research and Reference Program) was propagated in H9 cells at 37° C., 5% CO₂ in RPMI 1640 medium supplemented with 10% heat inactivated fetal calf serum and standard antibiotics. The culture supernatants was filtered and aliquotted. MT4 cells were incubated with virus (0.005 MOI) and growth medium containing dilutions of the test compounds for six days in parallel with virus-infected as well as uninfected control cultures without added compounds. Compound and buffers were also tested in parallel for cytotoxic effects in uninfected MT4 cultures. Both antiviral activity and cytotox tests were set up in duplicate. Test-compounds mediating less than 30% reduction of HIV-1 expression were considered without biological activity, whereas a 30% inhibition of cell growth relative to control cultures was considered significant and the 50% inhibitory concentration (IC50) was determined by interpolation from the plots of percent inhibition versus concentration of test compound. A representative plot of percent inhibition of growth versus concentration of BPFI-0300 is shown in FIG. 4.

A minor cytotoxic effect of the Tris-HCl pH 7.5 buffer was observed in the lower dilutions of the BPFI compounds. All BPFI fusion proteins exhibited an antiviral activity similar or slightly better compared to the T-20 compound. The interpolated IC50 values were as follows: T-20: 11 nM; BPFI-0100: 12 nM; BPFI-0200: 11 nM; BPFI-0300: 8 nM; and PFI-0400: 10 nM.

### EXAMPLE 5

Head to Head Toxicokinetic Analysis of HIV gp41 HR2 Derivatives BPFI-0101, -0201, -0301, and -0401 and T-20 (Fuzeon, Enfuvirtide) in Rats

Trimeric BPFI-0101 (SEQ ID NO 42), BPFI-0201 (SEQ ID NO 43), BPFI-0301 (SEQ ID NO 44) and monomeric BPFI-0401 fusion proteins were expressed in E. coli, purified, and processed essentially as described in Example 2 and 3. The synthetic peptide T-20 (Roche) and the BPFI fusion proteins were gelfiltrated into 0.1 M Borate buffer pH 9.0 for “Bolton-Hunter” 125I labelling on lysine residues using the standard reagent from Amersham-Biosciences as described by the supplier. After the labelling reaction the BPFI fusion proteins and the T-20 peptide were gelfiltrated into 20 mM Sodium Carbonate pH 9.0 and mannitol added to 0.1 M. The specific activity of the 125I labelled compounds were adjusted 5 μCi/mg with the corresponding unlabelled compound in 20 mM Sodium Carbonate pH 9.0, 100 mM mannitol.

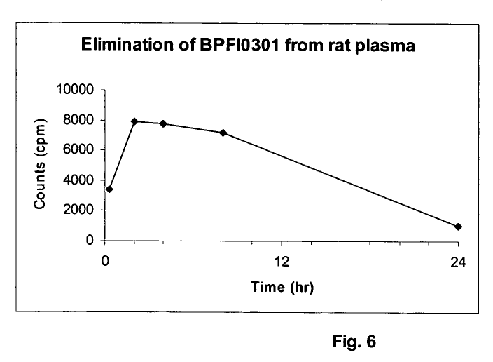

For the toxicokinetic analysis a total of 44 rats (22 male and 22 female) were placed in 5 groups (4 male and 4 female) and one control group (2 male and 2 female). Each of the 5 groups was subcutaneously injected a 5 mg/kg dose of one of the radio-labelled test compounds, BPFI-0101, -0201, -0301, -0401, or T-20 peptide, respectively, at time zero. The control group was only injected the vehicle buffer (20 mM Sodium Carbonate pH 9.0 and 0.1 M Mannitol). All animals were bled at time points: 20 min, 2, 5, 8, 24, 48, and 72 hours post injection. Samples were collected in EDTA containing tubes and plasma collected using standard procedures. The amount of radioactivity was measured and a clearing curve for each test compound plotted and pharmacokinetic parameters of the different compounds calculated. A clearing curve for compound BPFI-0301 is shown in FIG. 6.

The calculated elimination half-lives for the different compounds were as follows: BPFI-0101 12 hrs; BPFI-0201 10 hrs; BPFI-0301 9 hrs; BPFI-0401 2 hrs; and T-20 2 hrs. These results clearly demonstrate that the trimerised HR2 fragments (BPFI-0101, BPFI-0201, BPFI-0301) all have an elimination half-life which is significantly longer than the monomeric HR2 fragments T-20 and BPFI-0401.

The ratios between the observed “Area Under the Curve” (AUC) for the different BPFI compounds and the calculated AUC for T-20 were as follows: BPFI-0101:T-20=1.2; BPFI-0201:T-20=1.8; BPFI-0301:T-20=3.5; and BPFI-0401:T-20=1.0 (AUC was calculated using WinNolin version 4.1, Pharsight Corporation). As can be seen from these results, the monomeric HR2 fragment BPFI-0401 has an AUC value which is the same as the AUC found for T-20. In contrast hereto, the trimeric HR2 fragments BPFI-0101, BPFI-0201 and BPFI-0301 all have an AUC value which is higher than for T-20. In particular it should be noted that the trimeric HR2 fragment BPFI-0301 has an AUC, and thereby a bioavailability, which is 3.5 times higher than T-20 and the monomeric BPFI-0401. Surprisingly, BPFI-0301 is trimerised using the 37 amino acid trimerisation module (TTSE) fragment (V17; SEQ ID NO 65) which was the shortest of the applied TTSE's. The TTSE's applied in BPFI-0101 and BPFI-0201 contained 53 amino acids (E1; SEQ ID NO 50) and 44 amino acids (110; SEQ ID NO 59), respectively. This suggests that it may be other factors than the actual size of the protein constructs which influence the degradation of the compounds and thereby the AUC values of the compounds.

All animals were killed at the end of the experiment (72 hours post injection) and the kidneys, livers and skin (injection area) inspected for abnormalities. No adverse reactions were observed.

### EXAMPLE 6

Formulation of BPFI-0301 at Physiologic pH and Osmolarity

Trimeric BPFI-0301 (125 mg) produced in E. coli and processed as described in Example 3 was gelfiltrated into a buffer containing 100 mM NH₄HCO₃ adjusted to pH 7.2 with CO₂ and lyophilised. After lyophilisation BPFI-0301 was dissolved into 5 mL of 100 mM NH₄HCO₃ pH 7.4 and 20 mM mannitol, transferred to a new and smaller glass container, and re-lyophilised. The lyophilised material was readily dissolved (within 15 min) into one mL of a buffer containing 20 mM NaHCO₃ pH 7.4. The concentration of BPFI-0301 in solution was determined to 110 mg/mL.

### REFERENCES

Bray, B. L. Large-scale manufacture of peptide therapeutics by chemical synthesis. Nature Reviews Drug Discovery, 2003, vol. 2, p. 587-593.
Fuzeon label, Roche Pharmaceuticals, 2003.
Hochuli, et al. Biotechnology, 1988, p. 1321-1325.
Kilby, J. M., et al. Novel Therapies based on Mechanisms of HIV 1 cell entry. New England Journal of Medicine, 2003, vol. 348, no.22, p. 2228 2238.
Levin, Jules. Update on Fuzeon and T1249 fusion inhibitor halted development. NATAP, 01.07.2004.
Moore, J. P., et al. The entry of entry inhibitors: A fusion of science and medicine. PNAS, 2003, vol. 100, no.19, p. 10598-10602.
Root, M. J., et al. Protein Design of an HIV-1 Entry Inhibitor. Science, 2001, vol. 291, p. 884-888.
Russell et al. J. AM. ACAD. DERMATOL., 2003, Vol. 49(5), pp. 826-831.
Steinbrook, R. HIV infection-A new Drug and New Costs. The New England Journal of Medicine, 2003, vol. 348, no.22, p. 2171-2172.
Studier, et al. Journal of Molecular Biology, 1986, vol 189, p. 113-130.

1. A fusion protein exhibiting anti-viral activity comprising: (i) a first polypeptide representing the HR2 region of the ectodomain of the human immunodeficiency virus gp41 protein or a part thereof, and (ii) a second polypeptide representing a multimerisation domain peptide.

2. A fusion protein according to claim 1, wherein the multimerisation domain peptide is selected from the group consisting of a dimerising domain, a trimerising domain, a tetramerising domain, a pentamerising domain and a hexamerising domain.

3. A fusion protein according to claim 1, wherein said first polypeptide is linked to the N-terminal amino acid residue of the multimerisation domain peptide.

4. A fusion protein according to claim 1, wherein said first polypeptide is linked to the C-terminal amino acid residue of the multimerisation domain peptide.

5. A fusion protein according to claim 1, wherein the multimerisation domain is a trimerising domain derived from tetranectin.

6. A fusion protein according to claim 5, wherein the trimerising domain derived from tetranectin comprises a sequence having at least 68% amino acid sequence identity with the sequence of SEQ ID NO 2.

7. A fusion protein according to claim 5, wherein the amino acid sequence identity is at least 75%.

8. A fusion protein according to claim 5, wherein the trimerising domain derived from tetranectin comprises the amino acid sequence SEQ ID NO 2.

9. A fusion protein according to claim 5, wherein the trimerising domain derived from tetranectin comprises an amino acid sequence selected from the group consisting of SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72 and SEQ ID NO 73.

10. A fusion protein according to claim 1, wherein the first polypeptide representing the HR2 domain comprises the amino acid sequence of SEQ ID NO 1.

11. A fusion protein according to claim 1, wherein the first polypeptide representing the HR2 region comprises a fragment of the amino acid sequence of SEQ ID NO 1.

12. A fusion protein according to claim 11, wherein the number of amino acids in said fragment is in a range selected from the group consisting of 20-73 amino acids, 30-73 amino acids, 40-70 amino acids, 30-65 amino acids, 30-60 amino acids, 30-55 amino acids, 30-50 amino acids, 30-45 amino acids, 30-40 amino acids and 30-35 amino acids.

13. A fusion protein according to claim 11, wherein the fragment of SEQ ID NO 1 comprises SEQ ID NO 4, SEQ ID NO 5, SEQ ID NO 49 or SEQ ID NO 159.

14. A fusion protein according to claim 11, wherein the fragment of SEQ ID NO 1 is selected from the group consisting of SEQ ID Nos 74-115.

15. A fusion protein according to claim 11, wherein the fragment of SEQ ID NO 1 is selected from the group consisting of SEQ ID Nos 116-158.

16. A fusion protein according to claim 1, wherein the first polypeptide representing the HR2 domain comprises of an amino acid sequence selected from the group consisting of SEQ ID NO 6, SEQ ID NO 7, SEQ ID NO 8 and SEQ ID NO 9.

17. A fusion protein according to claim 1, wherein the human immunodeficiency virus is selected from the group consisting of HIV-1 and HIV-2.

18. A fusion protein according to claim 5, selected from the group consisting of BPFI-0100 (SEQ ID NO 10), BPFI-0200 (SEQ ID NO 11), BPFI-0300 (SEQ ID NO 12), BPFI-0101 (SEQ ID NO 42), BPFI-0201 (SEQ ID NO 43) and BPFI-0301 (SEQ ID NO 44).

19. A fusion protein according to claim 1, further comprising a linker between the first polypeptide and the second polypeptide.

20. A polypeptide complex comprising at least two fusion proteins according to claim 1.

21. A polypeptide complex comprising three fusion proteins according to claim 1.

22. A polypeptide complex according to claim 20 or 21, exhibiting an in-vitro antiviral activity against strain IIIB of HIV-1 using MT4 cells as target cells, with an 50% inhibitory concentration (IC50) in the range of 1-500 nM.

23. A method of treating HIV infection in a subject, comprising administering to the subject a therapeutically effective amount of the polypeptide complex according to claim 20 or 21.

24. A method according to claim 23, further comprising the administration of at least one further therapeutic agent.

25. A pharmaceutical composition comprising the fusion protein according to any of claims 1-19.

26. A method of producing a polypeptide complex according to claim 20 or 21, said method comprising the steps of (i) expressing or synthesizing a fusion protein exhibiting anti-viral activity, wherein said fusion protein comprises (a) a first polypeptide representing the HR2 region of the ectodomain of the human immunodeficiency virus gp41 protein or a part thereof, and (b) a second polypeptide representing a multimerisation domain peptide, (ii) effecting complex formation between said fusion proteins and, (iii) isolating the resulting polypeptide complex and optionally subjecting said polypeptide complex to further processing.

27. A method according to claim 26, wherein the fusion protein comprises a third fusion partner.

28. A method according to claim 27, wherein the third fusion partner is ubiquitin.

29. A method according to claim 27, wherein the junction region between said third fusion partner and the fusion protein comprises a Granzyme B protease cleavage site.

30. A method of inhibiting human and non-human retroviral transmission to uninfected cells comprising administering the fusion protein according to any of claims 1-19 to a subject in need thereof.

31. A method of preparing a pharmaceutical composition comprising associating the fusion protein according to any of claims 1-19 with a pharmaceutically acceptable carrier.

32. A composition comprising a fusion protein according to any of claims 1-19.

33. An isolated nucleic acid sequence encoding the fusion protein according to any of claims 1-19.

34. A recombinant vector comprising the isolated nucleic acid sequence according to claim 33.

35. A host cell transformed with a vector according to claim 34.

36. A fusion protein according to claim 5, wherein the amino acid sequence identity is at least 87%.

37. A fusion protein according to claim 5, wherein the amino acid sequence identity is at least 92%.

38. A polypeptide complex comprising at least three fusion proteins according to claim 1.

39. A polypeptide complex comprising at least four fusion proteins according claim 1.

40. A polypeptide complex comprising at least five fusion proteins according to claim 1.

41. A polypeptide complex comprising at least six fusion proteins according to claim 1.

42. A polypeptide complex according to claim 20 or 21, exhibiting an in-vitro antiviral activity against strain IIIB of HIV-1 using MT4 cells as target cells, with an 50% inhibitory concentration (IC50) in the range of 1-400 nM.

43. A polypeptide complex according to claim 20 or 21, exhibiting an in-vitro antiviral activity against strain IIIB of HIV-1 using MT4 cells as target cells, with an 50% inhibitory concentration (IC50) in the range of 1-300 nM.

44. A polypeptide complex according to claim 20 or 21, exhibiting an in-vitro antiviral activity against strain IIIB of HIV-1 using MT4 cells as target cells, with an 50% inhibitory concentration (IC50) in the range of 1-200 nM.

45. A polypeptide complex according to claim 20 or 21, exhibiting an in-vitro antiviral activity against strain IIIB of HIV-1 using MT4 cells as target cells, with an 50% inhibitory concentration (IC50) in the range of 1-100 nM.

46. A polypeptide complex according to claim 20 or 21, exhibiting an in-vitro antiviral activity against strain IIIB of HIV-1 using MT4 cells as target cells, with an 50% inhibitory concentration (IC50) in the range of 1-50 nM.

47. A polypeptide complex according to claim 20 or 21, exhibiting an in-vitro antiviral activity against strain IIIB of HIV-1 using MT4 cells as target cells, with an 50% inhibitory concentration (IC50) in the range of 1-40 nM.

48. A polypeptide complex according to claim 20 or 21, exhibiting an in-vitro antiviral activity against strain IIIB of HIV-1 using MT4 cells as target cells, with an 50% inhibitory concentration (IC50) in the range of 1-30 nM.

49. A polypeptide complex according to claim 20 or 21, exhibiting an in-vitro antiviral activity against strain IIIB of HIV-1 using MT4 cells as target cells, with an 50% inhibitory concentration (IC50) in the range of 1-20 nM.

50. A polypeptide complex according to claim 20 or 21, exhibiting an in-vitro antiviral activity against strain IIIB of HIV-1 using MT4 cells as target cells, with an 50% inhibitory concentration (IC50) in the range of 1-10 nM.

51. A pharmaceutical composition comprising the polypeptide complex according to claim 20 or 21.

52. A composition comprising a polypeptide complex according to claim 20 or 21.

53. A method of inhibiting human and non-human retroviral transmission to uninfected cells comprising administering the polypeptide complex according to claim 20 or 21 to a subject in need thereof.

54. A method of preparing a pharmaceutical composition comprising associating the polypeptide complex according to claim 20 or 21 with a pharmaceutically acceptable carrier.
